(19) United States
(12) Patent Application Publication
(10) Pub. No.: US 2026/0013968 A1
(43) Pub. Date: Jan. 15, 2026

(54) AUGMENTATION OVERLAY DEVICE

(71) Applicant: BRAINLAB AG, Münich (DE)

(72) Inventor: Dominik WEGERTSEDER, Munich (DE)

(21) Appl. No.: 18/992,666

(22) PCT Filed: Jul. 11, 2022
(86) PCT No.: PCT/EP2022/069287

§ 371 Date: Jan. 9, 2025

(51) Int. Cl. A61B 90/00; A61B 34/10; H04L 9/32

(52) U.S. Cl. CPC A61B 90/37; A61B 34/10; A61B 90/361; H04L 9/3271; A61B 2034/105; A61B 2090/365

(57) ABSTRACT

Provided are an augmentation overlay device and technique. The augmentation overlay device includes an image source configured for providing live image data of a region of interest in the form of a live image pixel stream; a data server configured for providing overlay data; and a composer device that is a hardware device with function implemented in hardware and configured for: providing display data in the form of the live image pixel stream by overlaying the provided live image data with the provided overlay data; determining a restricted area in the provided live image data, the provided overlay data including restricted area overlay data associated with the restricted area and free overlay data associated with an area outside of the restricted area; and restricting overlay with the restricted area overlay data. The augmentation overlay device includes a display device configured for receiving and displaying the provided display data.

### FIELD OF THE INVENTION

The present invention relates to an augmentation overlay device with hardware based functional safety, in particular used in a digital operation room, as well as a method for augmentation overlay with hardware based functional safety.

### TECHNICAL BACKGROUND

An Augmentation/Video-composition product, also referred to as augmentation overlay device, generally allows a user to overlay a video stream with any kind of additional data. Thus, in the context of an operation room with digital support, also referred to as digital operation room, an augmentation overlay device can be very helpful to the medical personal. Augmentation overlay devices like the Buzz Virtual of the Brainlab AG overlays additional patient data, referred to as overlay data, onto a main video while simultaneously delivering on documentation needs on a display. This is done by processing the main video with a software, in particular by using a graphics card, However, hiding important areas of the main video with overlay data may result in harm to patients. Common systems consequently provide a software solution with a “overlay-off” button to deactivate all overlays so that the display only shows the main video. In case of a software error, this function might lead to an extended blocking of the main video with overlay data or in the worst case a complete shutdown of the displayed information. Such events at the wrong time of an operation might lead to significant harm of the patient. Alternatively, the display is provided with a bypass cable and the user can switch to the second input, but the source has to provide an additional output and the user has to find the input selector switch fast enough. Performing such actions during an operation is highly undesired.

The disadvantage of the current solution is that the software solution is not very safe. Software solutions basically bypass or disable the complete device, which result in additional installation/material effort and the need to train the user to enable the bypass. In several installation scenarios, this is difficult to implement. For example in a scenario, where no backup video output is available on the image source to switch to in the case of software failure or in a scenario, where the display is not in reach of the user (Ceiling mount, wall mount, technical room) no bypass is possible.

The present invention has the object of providing an augmentation overlay device, which safely prevents unintended overlay in the main video.

The present invention can be used for intra-operative augmentation overlay in digital operating room procedures e.g. in connection with a system for augmentation overlay like the Buzz Virtual of Brainlab AG.

Aspects of the present invention, examples and exemplary steps and their embodiments are disclosed in the following. Different exemplary features of the invention can be combined in accordance with the invention wherever technically expedient and feasible.

### EXEMPLARY SHORT DESCRIPTION OF THE INVENTION

In the following, a short description of the specific features of the present invention is given which shall not be understood to limit the invention only to the features or a combination of the features described in this section.

The invention relates to an augmentation overlay device, comprising the following. An image source, configured for providing live image data of a region of interest in form of a live image pixel stream. A data server, configured for providing overlay data. A composer device, wherein the composer device is a hardware device and is configured for providing display data in form of a live image pixel stream by overlaying the provided live image data with the provided overlay data. The composer device is configured for determining a restricted area in the provided live image data, wherein the provided overlay data comprises restricted area overlay data that is associated with the restricted area of the live image data and free overlay data that is associated with an area of the live image data outside of the restricted area. The composer device is configured for restricting overlay with the restricted area overlay data. The augmentation overlay device further comprises a display device, configured for receiving and displaying the provided display data.

### GENERAL DESCRIPTION OF THE INVENTION

In this section, a description of the general features of the present invention is given for example by referring to possible embodiments of the invention.

The present invention is defined by the subject-matter of the independent claims. Additional features of the invention are presented in the dependent claims.

In a first aspect of the invention, an augmentation overlay device comprises an image source, configured for providing live image data of a region of interest in form of a live image pixel stream. The augmentation overlay device comprises a data server, configured for providing overlay data. The augmentation overlay device comprises a composer device. The composer device is a hardware device, wherein a function of the hardware device is implemented in hardware, and is configured for providing display data in form of a live image pixel stream by overlaying the provided live image data with the provided overlay data. The composer device is configured for determining a restricted area in the provided live image data. The provided overlay data comprises restricted area overlay data that is associated with the restricted area of the live image data and free overlay data that is associated with an area of the live image data outside of the restricted area. The composer device is configured for restricting overlay with the restricted area overlay data. The augmentation overlay device comprises a display device, configured for receiving and displaying the provided display data.

In other words, the composer device is a hardware circuit, which safely prevents unintended overlay in restricted areas, which are also referred to as special areas, of the live image shown on the display device. Thus, the patients safety is increased, in particular by functional safety measures.

The known approach uses software to protect the restricted areas, which is not considered as functional safety measure as any misbehaving software can hide important parts of the live video relating to the live video data or resolving this behaviour comprises a deactivation and/or restart of the whole software system.

The term “augmentation overlay device”, as used herein, relates to a device that is configured for overlaying a main video with overlay data. The main video, also referred to as display data, comprises a video, in particular a live video, of a patient that a user, in particular medical personal, is performing a medical workflow, in particular surgery. The main video consequently comprises important information for the user that the augmentation overlay device overlays with additional information, the overlay data.

The term “live image data”, as used herein, relates to image data, in particular of a region of interest of a patient, on which a medical workflow is performed. The live image data is preferably provided in form of a live image pixel stream by an image source, in particular a video camera, for example an endoscope or a microscope. During the medical workflow, inter alia the live image data is displayed to the user by a display device. The term “live”, as used herein, relates to a time delay, also referred to as latency, that is smaller than 100 us, wherein the time delay is defined by a time window between the time that the image source provides the live image pixel stream and the time that the display displays the corresponding live image pixel stream.

The term “overlay data”, as used herein, relates to any kind of auxiliary data that should help the user in performing the medical workflow.

The term “region of interest”, as used herein, relates to a region of the patient that the user is treated during the medical workflow.

The term “display device”, as used herein, relates to a display that for example is disposed in an operating room and is used by the user to visualize parts of the patient, for example provided by a digital endoscope or a digital microscope. For example, the display device is a smart display. Preferably, the display device is a ultra-HD display device.

The term “restricted area”, as used herein, relates to a portion of the live image data that is especially important for the user. In particular, the restricted area relates to that part of the live image data that shows the part of the patient that is treated by the user, for example an organ or a blood vessel. The restricted area is also referred to as special area or critical area. In other words, the region of interest covered by the live image comprises the part of the patient that is treated by the user, in general being a central portion of an image of the live image data. The region of interest preferably covers a larger area than the part of the patient that is treated by the user, leaving additional space, in particular at the borders of the region of interest, for auxiliary data.

The term “composer device”, as used herein, relates to a composer component, which is also referred to as mixer component, implemented in hardware. In other words, the composer device is configured to mix incoming live image data and overlay data to provide the respective display data that is shown on the display, illustrating the live video of the live image data and the auxiliary data of the overlay data overlaying the live video. In other words, the composer device ingests the incoming real-time pixels of a video stream (live image data) and composes an output pixel (display data) from the input (live image data) and the overlays (overlay data).

The term “hardware device”, as used herein, relates to a device that is configured to perform its function using a series of logic blocks. A logic block comprises a circuitry of a plurality of logic gates. In contrast, a software device that might comprise logic blocks to some extend, its main function is performed by a central processing unit, CPU. Examples for hardware devices comprise a field programmable gate array, FPGA, and an application specific integrated circuit, ASIC. The CPU in a software device executes a program sequentially. The hardware device does not execute a program in the software sense but executes its function as defined by the circuitry of logic blocks of the hardware device, allowing parallel system architecture with compared to software significantly higher speed. Furthermore, not running any software, the hardware device is not prone to software errors and as such more reliable than software applications.

The augmentation overlay device is preferably part of a digital operation room. The digital operation room comprises a plurality of devices that should help the user, in particular the surgeon, to perform a medical workflow on a patient. The user for example uses a digital endoscope providing live image data. The live image data is displayed on a display in the operation room, so that the user can watch his movement on the patient on the display, in particular in a magnified way. The live image data is further processed by the composer device to overlay the live image data with overlay data, also referred to as auxiliary data, providing additional information for the user on the display within the live image data.

The software approach handles each pixel of the live image pixel stream equally, wherein if the software produces an overlay over a restricted area of the live image data, or in other words the live video according to the live image data. The hardware based composer device is configured to restrict overlay of pixel areas of the live image pixel stream, providing functional safety.

The hardware approach of the composer device allows processing the overlay data on the live pixel stream, and consequently without using any frame buffers or similar representations of a frame of the live image data in a memory that a software would need to use. Consequently, the hardware composer device is not prone to software errors. This provides an overlay of live image data with improved reliability.

This is achieved by the following structure: The live video is feed to the mixer as a live stream of Pixels. The mixer parses the stream of pixels and synchronizes to it in terms of start of picture and start of lines positions in the pixel stream. With this synchronisation mechanism it prefetches a line of overlay data to internal RAM before it is needed from the slower main DRAM. This allows to do the overlay calculation from the received live video pixels and the according overlay pixel information from the prefetch buffer in real time.

Furthermore, as the hardware composer device directly works on the live image pixel stream, the overlay can be provided in real-time, in particular under 100 us, providing an overlay of the live image data with improved latency, in particular also in ultra-HD video scenarios. In other words, the display data can also referred to as live display data, as due to the execution of the overlay directly on the live image data, the display data has a latency to the live image pixel stream of under 100 us.

This allows to provide a hardware based real-time overlay of live image data with improved safety.

In a preferred embodiment, each of the pixels of the live image pixel stream of the live image data comprises x-y-coordinates. The restricted area of the live image pixel stream of the live image data is determined using predetermined restricted x-y-coordinates.

In other words, the composer device is provided with x-y-coordinates of the region of interest and with the x-y-coordinates of the region of the interest relating to the restricted area. As each pixel of the live image pixel stream comprises x-y-coordinates of the region of interest, the composer device can determine pixel by pixel if the pixel is part of the restricted area or not and can apply the restriction if necessary.

In a preferred embodiment, the restricted area comprises a shape. A dimension of the shape and a location of the shape in the region of interest is at least partly defined by the x-y-coordinates.

Preferably, the shape comprises a rectangular, a square, a circle and/or an ellipse. Further preferably, the shape of the restricted area is predetermined, in particular on demand or fixed in a hardware implementation. Alternatively, the shape of the restricted area is selectable, in particular by a user.

Consequently, the composer device is provided with a predetermined dimension of the shape, in particular type and size of the shape, of the restricted area together with the location of the shape in the live image data. The location of the shape preferably comprises at least one anchor point, in particular in x-y-coordinates. For example, the location of the shape comprises a center point of a circle shape, wherein the dimension of the shape indicates that the shape is a circle and that the diameter is 1000 pixel.

Consequently, the user can easily define the restricted area that can be interpreted by the composer device using the x-y-coordinates of the pixels of the live image pixel stream.

In a preferred embodiment, the restricted area of the live image pixel stream of the live image data is determined using a mask layer.

Preferably, the mask layer is implemented as a random access memory, RAM, or RAM region. Each storage element of the RAM or RAM region comprises the restriction, or in other words restriction level, of a pixel (or group of pixels for lower granularity) of the live image stream, defining the restricted area.

In a preferred embodiment, the composer device comprises a mixing unit that is configured for providing the display data in form of a live image pixel stream by overlaying the provided live image data with the provided overlay data. The mixing unit is configured for not reading the restricted area overlay data.

In other words, the mixing unit checks each pixel of the overlay data, whether it is part of the restricted area or not. Consequently, each part of the overlay data that refers to the restricted area is not further processed, or in other words not read. Thus, only the overlay data that refers not to the restricted area is stored in a read access memory, RAM, of the augmentation overlay device to be mixed with the live image pixel stream by the mixing unit. Consequently, less memory transactions have to be performed on the RAM (or virtual RAM) of the augmentation overlay device when determining the display data.

In a preferred embodiment, the composer device comprises a mixing unit that is configured for providing the display data in form of a live image pixel stream by overlaying the provided live image data with the provided overlay data. The composer device is configured for discarding the restricted area overlay data.

In other words, the mixing unit checks each pixel of the overlay data, whether it is part of the restricted area or not after it has been read into the memory, in particular the RAM, of the augmentation overlay device. Consequently, each part of the overlay data that refers to the restricted area is just discarded, or in other words deleted from the memory. Thus, only the overlay data that refers not to the restricted area is mixed with the live image pixel stream by the mixing unit.

In a preferred embodiment, the composer device comprises a mixing unit that is configured for providing the display data in form of a live image pixel stream by overlaying the provided live image data with the provided overlay data. The composer device is configured for determining a transparency/alpha value of the restricted area overlay data.

Preferably, the composer device is configured for limiting the transparency/alpha value of the restricted area overlay data to a predetermined minimum value. Further preferably, the minimum value is smaller than 50% transparency. Further preferably, the minimum value is smaller than 20% transparency.

Preferably, the transparency value is provided by the user, in particular via an interactive menu displayed on the display device.

In a preferred embodiment, the composer device is configured for only considering the restricted area overlay data, when an additional security procedure is successfully performed.

Preferably, the term “considering” the overlay data, as used herein, relates to reading or not discarding the overlay data as described above.

The term “security procedure”, as used herein, relates to an additional security measure implemented to allow enabling overlaying the restricted area with overlay data without restrictions or outside of the aforementioned transparency levels.

In some scenarios, full overlay of the live image data might be desired. For example, augmented reality navigation functions or augmented reality 3D functions inherently require overlaying the respective part of the patient in the live image stream. Thus, enabling overlaying the restricted area with overlay data outside of the aforementioned restrictions is secured by additional security measures, referred to as security procedure.

In a preferred embodiment, the security procedure comprises a predetermined specific user interaction performed by a user.

In other words, the restricted area overlay data are only considered after an allowance by a user interaction. Preferably, the augmentation overlay device comprises a hardware switch that can be activated by user interaction and activates/deactivates considering the restricted area overlay data. The hardware switch can be used by the user directly at the augmentation overlay device and/or be used by the user over the user interface displayed in a non restricted area of the live image data.

In a preferred embodiment, the security procedure comprises a complex enabling sequence.

This means that it is not enough to flip a bit by software to enable or disable overlay, but have a more secure way to avoid unwanted activation. This preferably comprises to have a “magic” sequence of bytes to write to a register (eg. 4711) or to have a changing pattern (eg. 1st time 4711, 2nd time 4712 . . . ) or expect a correct calculation (eg. Read a number from a register, multiply by 3 and write it back to a register and only if this is correct the activation takes place).

Consequently, accidently unrestricted overlay of the live image data can be prevented.

In a preferred embodiment, the complex enabling sequence comprises a challenge response from the data server providing the overlay data.

In other words, a challenge response from software to the hardware of the composer device is necessary. This is similar to a challenge response authentication: A challenge “string” is requested by the software from the safety overlay device. The software calculates a response with a security “certificate”, in particular only available in authorized software parts and check in distributed software parts for the legitimacy of the request. This response is then sent to the augmentation overlay device and checked there for correct response and only in correct respond cases the restricted area overlay data is considered.

In a preferred embodiment, the security procedure comprises a request for overlay that enables the composer device to consider the restricted area overlay data for a predetermined limited time and disables overlay automatically if no security procedure is passed before the limited time times out.

In other words, unrestricted area overlay data is only considered for limited time only. Consequently, the request for overlay has to be repeated regularly.

The value for the limited time is either predetermined in the hardware of the composer device or the user can input the value for the limited time over the user interface displayed in a non restricted area of the live image data.

Preferably, the user can input the request for overlay over the user interface displayed in a non restricted area of the live image data.

In a preferred embodiment, the image source comprises a medical imaging device.

Preferably, the image source is an endoscope or a microscope, in particular a digital endoscope or a digital microscope.

In a preferred embodiment, the overlay data comprises sensor data, patient monitoring data, 3D visualisation data, overlay menu data and/or videoconference data.

The overlay data for example comprise patient monitoring data, which indicate vital signs of the patient. The overlay data for example comprise 3D visualisation data, which indicate 3D menus or 3D extensions of 2D data of the live image data. The overlay data for example comprise patient security data, which indicate navigation data or marker data of a surgical navigation system used in the medical workflow. The overlay data for example comprise overlay menu data, indicating interactive menus and user inputs of the overlay data. The overlay data for example comprise video conference data, indicating a video feed of a second user to communicate with the user performing the medical workflow. The overlay data for example comprise sensor fusion data, indicating pre-operative and/or intra-operative sensor images, like computer tomography images or ultra sound images. The overlay data for example comprise telestration data, which is meant to have the video data transferred to an additional doctor, in particular to get a 2^{nd} opinion. The additional doctor can markup areas in the video and the markup is then shown on the main doctors video screen on the live video as augmentation.

In a preferred embodiment, the composer device is implemented at least partially in an ASIC or FPGA.

Preferably, the composer device is completely implemented in an ASIC or FPGA. The function of the composer device is preferably described by a hardware description language, HDL, for example very high speed integrated circuit hardware description language, VHDL, or Verilog.

In a second aspect of the invention, a method for augmentation overlay comprises the following steps. Providing, by an image source, live image data of a region of interest in form of a live image pixel stream. Providing, by a data server, overlay data.

Providing, by a composer device being a hardware device, wherein a function of the hardware device is implemented in hardware, display data in form of a live image pixel stream by overlaying the provided live image data with the provided overlay data, comprising determining, by the composer device, a restricted area in the provided live image data. The provided overlay data comprises restricted area overlay data that is associated with the restricted area of the live image data and free overlay data that is associated with an area of the live image data outside of the restricted area. The method further comprises the following step: Restricting, by the composer device, overlay with the restricted area overlay data. Displaying, by a display device the provided display data.

For example, the invention does not involve or in particular comprise or encompass an invasive step which would represent a substantial physical interference with the body requiring professional medical expertise to be carried out and entailing a substantial health risk even when carried out with the required professional care and expertise.

For example, the invention does not comprise a step of positioning a medical implant in order to fasten it to an anatomical structure or a step of fastening the medical implant to the anatomical structure or a step of preparing the anatomical structure for having the medical implant fastened to it. More particularly, the invention does not involve or in particular comprise or encompass any surgical or therapeutic activity. The invention is instead directed as applicable a digital operation room, wherein an augmentation overlay device with hardware based functional safety overlays additional patient data onto a live video stream while simultaneously delivering on documentation needs. For this reason alone, no surgical or therapeutic activity and in particular no surgical or therapeutic step is necessitated or implied by carrying out the invention.

The present invention also relates to the use of the augmentation overlay device or any embodiment thereof in a digital operation room, in particular for overlaying additional patient data onto a live video stream while simultaneously delivering on documentation needs.

### DEFINITIONS

In this section, definitions for specific terminology used in this disclosure are offered which also form part of the present disclosure.

### Marker

It is the function of a marker to be detected by a marker detection device (for example, a camera or an ultrasound receiver or analytical devices such as CT or MRI devices) in such a way that its spatial position (i.e. its spatial location and/or alignment) can be ascertained. The detection device is for example part of a navigation system. The markers can be active markers. An active marker can for example emit electromagnetic radiation and/or waves which can be in the infrared, visible and/or ultraviolet spectral range. A marker can also however be passive, i.e. can for example reflect electromagnetic radiation in the infrared, visible and/or ultraviolet spectral range or can block x-ray radiation. To this end, the marker can be provided with a surface which has corresponding reflective properties or can be made of metal in order to block the x-ray radiation. It is also possible for a marker to reflect and/or emit electromagnetic radiation and/or waves in the radio frequency range or at ultrasound wavelengths. A marker preferably has a spherical and/or spheroid shape and can therefore be referred to as a marker sphere; markers can however also exhibit a cornered, for example cubic, shape.

### Marker Device

A marker device can for example be a reference star or a pointer or a single marker or a plurality of (individual) markers which are then preferably in a predetermined spatial relationship. A marker device comprises one, two, three or more markers, wherein two or more such markers are in a predetermined spatial relationship. This predetermined spatial relationship is for example known to a navigation system and is for example stored in a computer of the navigation system.

In another embodiment, a marker device comprises an optical pattern, for example on a two-dimensional surface. The optical pattern might comprise a plurality of geometric shapes like circles, rectangles and/or triangles. The optical pattern can be identified in an image captured by a camera, and the position of the marker device relative to the camera can be determined from the size of the pattern in the image, the orientation of the pattern in the image and the distortion of the pattern in the image. This allows determining the relative position in up to three rotational dimensions and up to three translational dimensions from a single two-dimensional image.

The position of a marker device can be ascertained, for example by a medical navigation system. If the marker device is attached to an object, such as a bone or a medical instrument, the position of the object can be determined from the position of the marker device and the relative position between the marker device and the object. Determining this relative position is also referred to as registering the marker device and the object. The marker device or the object can be tracked, which means that the position of the marker device or the object is ascertained twice or more over time.

### Marker Holder

A marker holder is understood to mean an attaching device for an individual marker which serves to attach the marker to an instrument, a part of the body and/or a holding element of a reference star, wherein it can be attached such that it is stationary and advantageously such that it can be detached. A marker holder can for example be rod-shaped and/or cylindrical. A fastening device (such as for instance a latching mechanism) for the marker device can be provided at the end of the marker holder facing the marker and assists in placing the marker device on the marker holder in a force fit and/or positive fit.

### Pointer

A pointer is a rod which comprises one or more-advantageously, two-markers fastened to it and which can be used to measure off individual co-ordinates, for example spatial co-ordinates (i.e. three-dimensional co-ordinates), on a part of the body, wherein a user guides the pointer (for example, a part of the pointer which has a defined and advantageously fixed position with respect to the at least one marker attached to the pointer) to the position corresponding to the co-ordinates, such that the position of the pointer can be determined by using a surgical navigation system to detect the marker on the pointer. The relative location between the markers of the pointer and the part of the pointer used to measure off co-ordinates (for example, the tip of the pointer) is for example known. The surgical navigation system then enables the location (of the three-dimensional co-ordinates) to be assigned to a predetermined body structure, wherein the assignment can be made automatically or by user intervention.

### Reference Star

A “reference star” refers to a device with a number of markers, advantageously three markers, attached to it, wherein the markers are (for example detachably) attached to the reference star such that they are stationary, thus providing a known (and advantageously fixed) position of the markers relative to each other. The position of the markers relative to each other can be individually different for each reference star used within the framework of a surgical navigation method, in order to enable a surgical navigation system to identify the corresponding reference star on the basis of the position of its markers relative to each other. It is therefore also then possible for the objects (for example, instruments and/or parts of a body) to which the reference star is attached to be identified and/or differentiated accordingly. In a surgical navigation method, the reference star serves to attach a plurality of markers to an object (for example, a bone or a medical instrument) in order to be able to detect the position of the object (i.e. its spatial location and/or alignment). Such a reference star for example features a way of being attached to the object (for example, a clamp and/or a thread) and/or a holding element which ensures a distance between the markers and the object (for example in order to assist the visibility of the markers to a marker detection device) and/or marker holders which are mechanically connected to the holding element and which the markers can be attached to.

### Navigation System

The present invention is also directed to a navigation system for computer-assisted surgery. This navigation system preferably comprises the aforementioned computer for processing the data provided in accordance with the computer implemented method as described in any one of the embodiments described herein. The navigation system preferably comprises a detection device for detecting the position of detection points which represent the main points and auxiliary points, in order to generate detection signals and to supply the generated detection signals to the computer, such that the computer can determine the absolute main point data and absolute auxiliary point data on the basis of the detection signals received. A detection point is for example a point on the surface of the anatomical structure which is detected, for example by a pointer. In this way, the absolute point data can be provided to the computer. The navigation system also preferably comprises a user interface for receiving the calculation results from the computer (for example, the position of the main plane, the position of the auxiliary plane and/or the position of the standard plane). The user interface provides the received data to the user as information. Examples of a user interface include a display device such as a monitor, or a loudspeaker. The user interface can use any kind of indication signal (for example a visual signal, an audio signal and/or a vibration signal). One example of a display device is an augmented reality device (also referred to as augmented reality glasses) which can be used as so-called “goggles” for navigating. A specific example of such augmented reality glasses is Google Glass (a trademark of Google, Inc.). An augmented reality device can be used both to input information into the computer of the navigation system by user interaction and to display information outputted by the computer.

### Surgical Navigation System

A navigation system, such as a surgical navigation system, is understood to mean a system which can comprise: at least one marker device; a transmitter which emits electromagnetic waves and/or radiation and/or ultrasound waves; a receiver which receives electromagnetic waves and/or radiation and/or ultrasound waves; and an electronic data processing device which is connected to the receiver and/or the transmitter, wherein the data processing device (for example, a computer) for example comprises a processor (CPU) and a working memory and advantageously an indicating device for issuing an indication signal (for example, a visual indicating device such as a monitor and/or an audio indicating device such as a loudspeaker and/or a tactile indicating device such as a vibrator) and a permanent data memory, wherein the data processing device processes navigation data forwarded to it by the receiver and can advantageously output guidance information to a user via the indicating device. The navigation data can be stored in the permanent data memory and for example compared with data stored in said memory beforehand.

### Landmarks

A landmark is a defined element of an anatomical body part which is always identical or recurs with a high degree of similarity in the same anatomical body part of multiple patients. Typical landmarks are for example the epicondyles of a femoral bone or the tips of the transverse processes and/or dorsal process of a vertebra. The points (main points or auxiliary points) can represent such landmarks. A landmark which lies on (for example on the surface of) a characteristic anatomical structure of the body part can also represent said structure. The landmark can represent the anatomical structure as a whole or only a point or part of it. A landmark can also for example lie on the anatomical structure, which is for example a prominent structure. An example of such an anatomical structure is the posterior aspect of the iliac crest. Another example of a landmark is one defined by the rim of the acetabulum, for instance by the centre of said rim. In another example, a landmark represents the bottom or deepest point of an acetabulum, which is derived from a multitude of detection points. Thus, one landmark can for example represent a multitude of detection points. As mentioned above, a landmark can represent an anatomical characteristic which is defined on the basis of a characteristic structure of the body part. Additionally, a landmark can also represent an anatomical characteristic defined by a relative movement of two body parts, such as the rotational centre of the femur when moved relative to the acetabulum.

### Imaging Geometry

The information on the imaging geometry preferably comprises information which allows the analysis image (x-ray image) to be calculated, given a known relative position between the imaging geometry analysis apparatus and the analysis object (anatomical body part) to be analysed by x-ray radiation, if the analysis object which is to be analysed is known, wherein “known” means that the spatial geometry (size and shape) of the analysis object is known. This means for example that three-dimensional, “spatially resolved” information concerning the interaction between the analysis object (anatomical body part) and the analysis radiation (x-ray radiation) is known, wherein “interaction” means for example that the analysis radiation is blocked or partially or completely allowed to pass by the analysis object. The location and in particular orientation of the imaging geometry is for example defined by the position of the x-ray device, for example by the position of the x-ray source and the x-ray detector and/or for example by the position of the multiplicity (manifold) of x-ray beams which pass through the analysis object and are detected by the x-ray detector. The imaging geometry for example describes the position (i.e. the location and in particular the orientation) and the shape (for example, a conical shape exhibiting a specific angle of inclination) of said multiplicity (manifold). The position can for example be represented by the position of an x-ray beam which passes through the centre of said multiplicity or by the position of a geometric object (such as a truncated cone) which represents the multiplicity (manifold) of x-ray beams. Information concerning the above-mentioned interaction is preferably known in three dimensions, for example from a three-dimensional CT, and describes the interaction in a spatially resolved way for points and/or regions of the analysis object, for example for all of the points and/or regions of the analysis object. Knowledge of the imaging geometry for example allows the location of a source of the radiation (for example, an x-ray source) to be calculated relative to an image plane (for example, the plane of an x-ray detector). With respect to the connection between three-dimensional analysis objects and two-dimensional analysis images as defined by the imaging geometry, reference is made for example to the following publications:
1. “An Efficient and Accurate Camera Calibration Technique for 3D Machine Vision”, Roger Y. Tsai, Proceedings of the IEEE Conference on Computer Vision and Pattern Recognition. Miami Beach, Florida, 1986, pages 364-374
2. “A Versatile Camera Calibration Technique for High-Accuracy 3D Machine Vision Metrology Using Off-the-Shelf TV Cameras and Lenses”, Roger Y. Tsai, IEEE Journal of Robotics and Automation, Volume RA-3, No. 4, August 1987, pages 323-344.
3. “Fluoroscopic X-ray Image Processing and Registration for Computer-Aided Orthopedic Surgery”, Ziv Yaniv
4. EP 08 156 293.6
5. US 61/054,187

### Shape Representatives

Shape representatives represent a characteristic aspect of the shape of an anatomical structure. Examples of shape representatives include straight lines, planes and geometric figures. Geometric figures can be one-dimensional such as for example axes or circular arcs, two-dimensional such as for example polygons and circles, or three-dimensional such as for example cuboids, cylinders and spheres. The relative position between the shape representatives can be described in reference systems, for example by co-ordinates or vectors, or can be described by geometric variables such as for example length, angle, area, volume and proportions. The characteristic aspects which are represented by the shape representatives are for example symmetry properties which are represented for example by a plane of symmetry. Another example of a characteristic aspect is the direction of extension of the anatomical structure, which is for example represented by a longitudinal axis. Another example of a characteristic aspect is the cross-sectional shape of an anatomical structure, which is for example represented by an ellipse. Another example of a characteristic aspect is the surface shape of a part of the anatomical structure, which is for example represented by a plane or a hemisphere. For example, the characteristic aspect constitutes an abstraction of the actual shape or an abstraction of a property of the actual shape (such as for example its symmetry properties or longitudinal extension). The shape representative for example represents this abstraction.

Analytical devices

The movements of the treatment body parts are for example due to movements which are referred to in the following as “vital movements”. Reference is also made in this respect to EP 2 189 943 A1 and EP 2 189 940 A1, also published as US 2010/0125195 A1 and US 2010/0160836 A1, respectively, which discuss these vital movements in detail. In order to determine the position of the treatment body parts, analytical devices such as x-ray devices, CT devices or MRT devices are used to generate analytical images (such as x-ray images or MRT images) of the body. For example, analytical devices are constituted to perform medical imaging methods. Analytical devices for example use medical imaging methods and are for example devices for analysing a patient's body, for instance by using waves and/or radiation and/or energy beams, for example electromagnetic waves and/or radiation, ultrasound waves and/or particles beams. Analytical devices are for example devices which generate images (for example, two-dimensional or three-dimensional images) of the patient's body (and for example of internal structures and/or anatomical parts of the patient's body) by analysing the body. Analytical devices are for example used in medical diagnosis, for example in radiology. However, it can be difficult to identify the treatment body part within the analytical image. It can for example be easier to identify an indicator body part which correlates with changes in the position of the treatment body part and for example the movement of the treatment body part. Tracking an indicator body part thus allows a movement of the treatment body part to be tracked on the basis of a known correlation between the changes in the position (for example the movements) of the indicator body part and the changes in the position (for example the movements) of the treatment body part. As an alternative to or in addition to tracking indicator body parts, marker devices (which can be used as an indicator and thus referred to as “marker indicators”) can be tracked using marker detection devices. The position of the marker indicators has a known (predetermined) correlation with (for example, a fixed relative position relative to) the position of indicator structures (such as the thoracic wall, for example true ribs or false ribs, or the diaphragm or intestinal walls, etc.) which for example change their position due to vital movements.

### Imaging Methods

In the field of medicine, imaging methods (also called imaging modalities and/or medical imaging modalities) are used to generate image data (for example, two-dimensional or three-dimensional image data) of anatomical structures (such as soft tissues, bones, organs, etc.) of the human body. The term “medical imaging methods” is understood to mean (advantageously apparatus-based) imaging methods (for example so-called medical imaging modalities and/or radiological imaging methods) such as for instance computed tomography (CT) and cone beam computed tomography (CBCT, such as volumetric CBCT), x-ray tomography, magnetic resonance tomography (MRT or MRI), conventional x-ray, sonography and/or ultrasound examinations, and positron emission tomography. For example, the medical imaging methods are performed by the analytical devices. Examples for medical imaging modalities applied by medical imaging methods are: X-ray radiography, magnetic resonance imaging, medical ultrasonography or ultrasound, endoscopy, elastography, tactile imaging, thermography, medical photography and nuclear medicine functional imaging techniques as positron emission tomography (PET) and Single-photon emission computed tomography (SPECT), as mentioned by Wikipedia.

The image data thus generated is also termed “medical imaging data”. Analytical devices for example are used to generate the image data in apparatus-based imaging methods. The imaging methods are for example used for medical diagnostics, to analyse the anatomical body in order to generate images which are described by the image data. The imaging methods are also for example used to detect pathological changes in the human body. However, some of the changes in the anatomical structure, such as the pathological changes in the structures (tissue), may not be detectable and for example may not be visible in the images generated by the imaging methods. A tumour represents an example of a change in an anatomical structure. If the tumour grows, it may then be said to represent an expanded anatomical structure. This expanded anatomical structure may not be detectable; for example, only a part of the expanded anatomical structure may be detectable. Primary/high-grade brain tumours are for example usually visible on MRI scans when contrast agents are used to infiltrate the tumour. MRI scans represent an example of an imaging method. In the case of MRI scans of such brain tumours, the signal enhancement in the MRI images (due to the contrast agents infiltrating the tumour) is considered to represent the solid tumour mass. Thus, the tumour is detectable and for example discernible in the image generated by the imaging method. In addition to these tumours, referred to as “enhancing” tumours, it is thought that approximately 10% of brain tumours are not discernible on a scan and are for example not visible to a user looking at the images generated by the imaging method.

### Medical Workflow

A medical workflow comprises a plurality of workflow steps performed during a medical treatment and/or a medical diagnosis. The workflow steps are typically, but not necessarily performed in a predetermined order. Each workflow step for example means a particular task, which might be a single action or a set of actions. Examples of workflow steps are capturing a medical image, positioning a patient, attaching a marker, performing a resection, moving a joint, placing an implant and the like.

### BRIEF DESCRIPTION OF THE DRAWINGS

In the following, the invention is described with reference to the appended figures, which give background explanations and represent specific embodiments of the invention. The scope of the invention is however not limited to the specific features disclosed in the context of the figures, wherein

FIG. 1a illustrates an augmentation overlay device with activated overlay in a restricted area;

FIG. 1b illustrates an augmentation overlay device with deactivated overlay in a restricted area;

FIG. 2 is a schematic illustration of composer device; and

FIG. 3 is a schematic illustration of a method for augmentation overlay.

### DESCRIPTION OF EMBODIMENTS

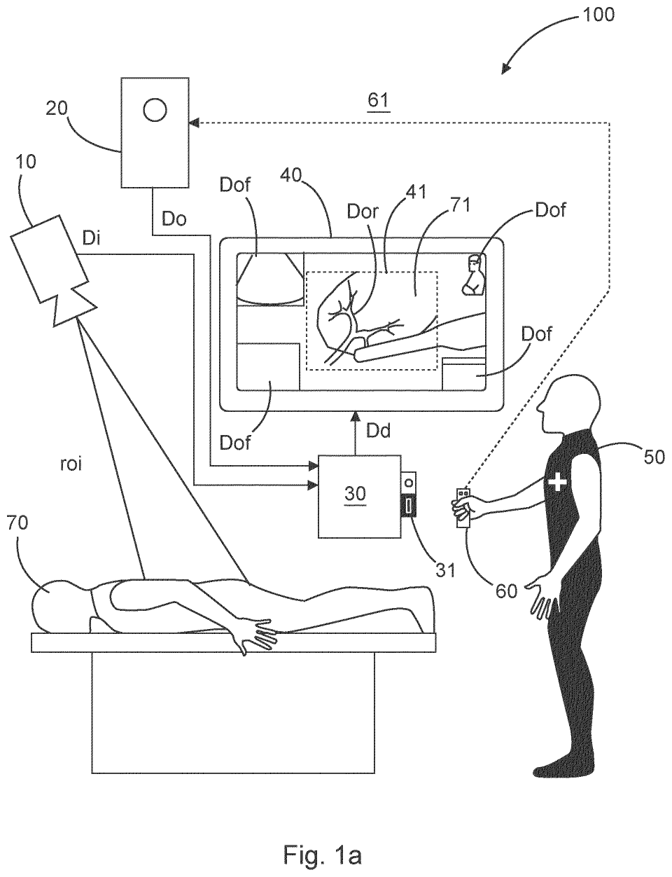

FIG. 1a illustrates an augmentation overlay device 100, comprising an image source 10, a data server 20, a composer device 30 and a display 40. Furthermore, FIG. 1a illustrates a user 50, in this case a surgeon, performing a medical workflow, in this case a surgery, at patient 70. The scenario shown in FIG. 1a relates to a digital operation room, in which the user 50 is supported by the augmentation overlay device 100. The image source 10 comprises a camera recording a region of interest roi of the patient 70. The region of interest roi is a portion of the patient 70 that is treated by the user 50. In his case, the user 50 treats an organ 71 of the patient 70. The image source 10 records the region of interest roi and provides live image data Di to the composer device 30. The data server 20 is configured for providing overlay data Do to the composer device 30. The composer device 30 is an ASIC/FPGA configured for determining display data Dd using the provided live image data Di and the overlay data Do. The live image data Di comprise a live image pixel stream, wherein the overlay data Do comprise information that should be displayed on the display 40 together with the live image data Di. Consequently, the display data Dd are basically the live image data Di overlaid with the overlay data Do.

The live image data Di comprise a live video captured by the image source 10 covering the region of interest roi. The live video show the organ that is treated by the user 50. The region around the organ is of critical interest for the user 50 and as such is defined as restricted area 41. The overlay of the live image data Di should be restricted in the restricted area 41 so that the user 50 is not restricted in his medical workflow.

The overlay data Do can be separated into restricted area overlay data Dor and free overlay data Dof.

The restricted area overlay data Dor relate to the restricted area. In this case, the restricted area overlay data Dor comprise an indicator for a blood vessel running through the organ 71. The restricted area overlay data Dor can be commonly known by the data server 20 due to the general structure of the organ 71 or can be provided externally by a sensor, like an X-ray device.

The free overlay data Dof relate to live image data Di outside of the restricted area 41. As illustrated, the free overlay data Dof comprise sensor data, in this case ultrasound data, patient monitoring data, menu data and/or videoconference data. The menu data indicate a user interface for the user 50 that helps the user 50 to control the composer device 30. The user 50 comprises an input device 60 that provides control data 61 to the data server 20, adjusting the overlay data Do.

The composer device 30 comprises an FPGA or an ASIC that executes the determining of the display data Dd by mixing the live image data Di with the overlay data Do. In this case, the composer device 30 also comprises an external hardware switch 31 that the user 50 can activate. In FIG. 1a, the switch is activated, allowing the composer device 30 to mix the live image data Di with the free overlay data Dof and the restricted area overlay data Dor. As such, the indicator of the blood vessel overlays the live image data Di in the restricted area 41.

The user 50 has activated the overlay of the restricted area 41, in particular via the user interface on the display 40. Nevertheless, the user 50 wants to prevent the overlay in the restricted area 41 promptly due to an unexpected emergency with the patient 70. Consequently, the user 50 can deactivate the external switch 31 of the composer device 30, preventing any overlay of the restricted area 41, which is indicated in FIG. 1b.

Thus, the free overlay data Dof is still used by the composer device 30 to overlay the live image data Di, however, the restricted area overlay data Dor is not considered anymore, leaving the restricted area 41 free of overlay.

Due to the composer device 30 being implemented in hardware, the overlay process is not prone to any software error and allows for a direct manipulation of the live image pixel stream, allowing for improved latency with delays under 100 us.

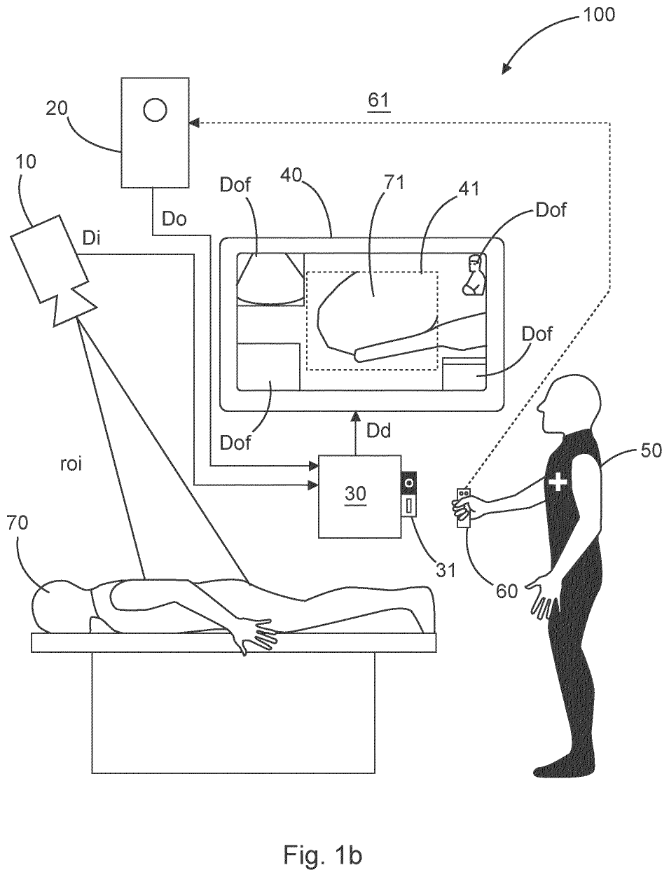

FIG. 2 is a schematic illustration of composer device 30. The composer device 30 comprises a mixing unit 32 that is configured to receive the live image data Di. The composer device 30 further comprises an overlay unit 33 that is configured to receive overlay data Do. Furthermore, the overlay unit 33 is configured to receive switching data Ds, in particular provided by a hardware switch 31. The switching data Ds indicates, if a restricted area 41 in the live image data Di is allowed to be overlaid by overlay data Do or not.

Consequently, if the switching data Ds indicates that all overlay is free, the overlay unit 33 provides free overlay data Dof as well as restricted area overlay data Dor of the overlay data Do to the mixing unit 32. The mixing unit 32 thus overlays the respective pixels of the live image data Di with the free overlay data Dof and the restricted area overlay data Dor. Otherwise, the overlay unit 33 only provides the free overlay data Dof to the mixing unit 32, which overlays the live image data Di with only the free overlay data Dof, inherently leaving the restricted area 41 free of overlay.

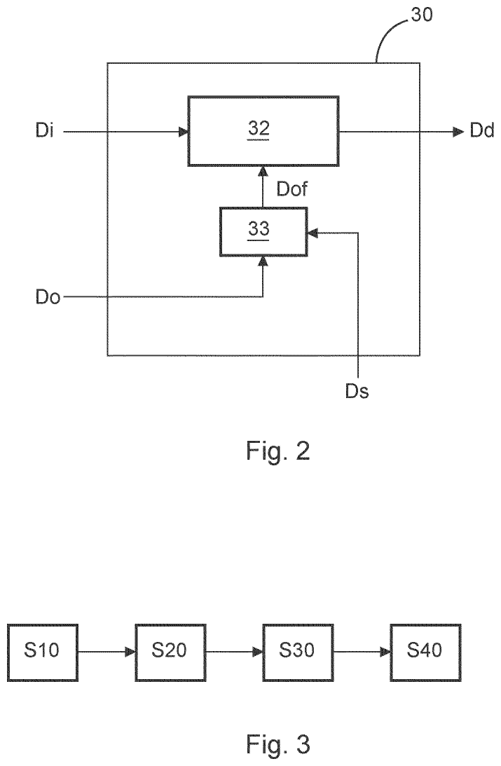

FIG. 3 is a schematic illustration of a method for augmentation overlay.

In a first step S10 an image source 10 provides live image data Di of a region of interest roi in form of a live image pixel stream. In a second step S20, a data server 20 provides overlay data Do. In a third step S30, a composer device 30, which is a hardware device, provides display data Dd in form of a live image pixel stream by overlaying the provided live image data Di with the provided overlay data Do, comprising determining, by the composer device 30 a restricted area 41 in the provided live image data Di, wherein the provided overlay data Do comprises restricted area overlay data Dor that is associated with the restricted area 41 of the live image data Di and free overlay data Dof that is associated with an area of the live image data Di outside of the restricted area 41, and restricting, by the composer device 30, overlay with the restricted area overlay data Dor. In a fourth step S40, a display device 40 receives and displays the provided display data Dd.

1. An augmentation overlay device, comprising:
an image source, configured for providing live image data of a region of interest in a form of a live image pixel stream;
a data server, configured for providing overlay data;
a composer device, wherein the composer device is a hardware device and a function of the hardware device is implemented in hardware,
wherein the composer device is configured for:
providing display data in the form of the live image pixel stream by overlaying the provided live image data with the provided overlay data;
determining a restricted area in the provided live image data, wherein the provided overlay data comprises restricted area overlay data that is associated with the restricted area of the live image data and free overlay data that is associated with an area of the live image data outside of the restricted area; and
restricting overlay via the restricted area overlay data; and
a display device, configured for receiving and displaying the provided display data.

2. The augmentation overlay device of claim 1, wherein each pixel of the live image pixel stream of the live image data comprises x-y-coordinates, wherein the restricted area of the live image pixel stream of the live image data is determined using predetermined restricted x-y-coordinates.

3. The augmentation overlay device of claim 2, wherein the restricted area comprises a shape, wherein a dimension and a location of the shape in the region of interest is at least partly defined by the x-y-coordinates.

4. The augmentation overlay device of claim 1, wherein the restricted area of the live image pixel stream of the live image data is determined using a mask layer.

5. The augmentation overlay device of claim 1, wherein the composer device comprises a mixing unit that is configured for:
providing the display data in the form of the live image pixel stream by overlaying the provided live image data with the provided overlay data; and
not reading the restricted area overlay data.

6. The augmentation overlay device of claim 1, wherein the composer device comprises a mixing unit that is configured for providing the display data in the form of the live image pixel stream by overlaying the provided live image data with the provided overlay data, and
wherein the composer device is further configured for;
discarding the restricted area overlay data; or
determining a transparency value/alpha value of the restricted area overlay data.

7. (canceled)

8. The augmentation overlay device of claim 1, wherein the composer device is configured for only considering the restricted area overlay data, when an additional security procedure is successfully performed.

9. The augmentation overlay device of claim 8, wherein the additional security procedure comprises a predetermined specific user interaction performed by a user.

10. The augmentation overlay device of claim 8, wherein the additional security procedure comprises a complex enabling sequence.

11. The augmentation overlay device of claim 10, wherein the complex enabling sequence comprises a challenge response from the data server providing the overlay data.

12. The augmentation overlay device of claim 8, wherein the additional security procedure comprises a request for overlay that enables the composer device to consider the restricted area overlay data for a predetermined limited time and to disable overlay automatically if no additional security procedure is passed before the limited time times out.

13. The augmentation overlay device of claim 1, wherein the image source comprises a medical imaging device.

14. The augmentation overlay device of claim 1, wherein the overlay data comprises one or more of: sensor data, patient monitoring data, 3D visualisation data, overlay menu data and/or videoconference data.

15. The augmentation overlay device of claim 1, wherein the composer device is implemented at least partially in an ASIC or FPGA.

16. A method for augmentation overlay, comprising:
providing, by an image source, live image data of a region of interest in a form of a live image pixel stream;
providing, by a data server, overlay data;
providing, by a composer device, which is a hardware device having a function implemented in hardware, display data in the form of the live image pixel stream by overlaying the provided live image data with the provided overlay data;
determining, by the composer device, a restricted area in the provided live image data, wherein the provided overlay data comprises restricted area overlay data that is associated with the restricted area of the live image data and free overlay data that is associated with an area of the live image data outside of the restricted area;
restricting, by the composer device, overlay with the restricted area overlay data; and
displaying, by a display device, the provided display data.

17. The method of claim 16, wherein each pixel of the live image pixel stream of the live image data comprises x-y-coordinates, the method further comprising:
determining, via predetermined restricted x-y-coordinates, the restricted area of the live image pixel stream of the live image data.

18. The method of claim 16, wherein the determining the restricted area comprises determining the restricted area in the provided live image data using a mask layer.

19. The method of claim 16, wherein the composer device comprises a mixing unit, the method further comprising:
providing, by the mixing unit, the display data in the form of the live image pixel stream by overlaying the provided live image data with the provided overlay data; and
not reading, by the mixing unity, the restricted area overlay data.

20. The method of claim 16, wherein the composer device comprises a mixing unit, the method further comprising:
providing, by the mixing unit, the display data in the form of the live image pixel stream by overlaying the provided live image data with the provided overlay data; and
discarding, by the composer device, the restricted area overlay data; or
determining, by the composer device, a transparency value/alpha value of the restricted area overlay data.

21. The method of claim 16, further comprising:
only considering, by the composer device, the restricted area overlay data when an additional security procedure is successfully performed,
wherein the additional security procedure comprises one or more of:
a predetermined specific user interaction performed by a user;
a complex enabling sequence, wherein the complex enabling sequence comprises a challenge response from the data server providing the overlay data; and/or
a request for overlay that enables the composer device to consider the restricted area overlay data for a predetermined limited time and to disable overlay automatically if no additional security procedure is passed before the limited time times out.
